(19) United States
(12) Patent Application Publication
(10) Pub. No.: US 2012/0171753 A1
(43) Pub. Date: Jul. 5, 2012

(54) SYSTEM AND METHOD FOR TREATING MATERIALS IN POOLS

(75) Inventor: Itai IVRY, Karkor (IL)

(73) Assignee: SEAMBIOTIC LTD., Tel Aviv (IL)

(21) Appl. No.: 13/226,187

(22) Filed: Sep. 6, 2011

(30) Foreign Application Priority Data
Dec. 30, 2010 (IL) 210376

(51) Int. Cl. C12N 1/12; C12M 1/02

(52) U.S. Cl. 435/257.3; 435/257.1; 435/289.1

(57) ABSTRACT

System and method are provided for treating materials in a liquid in a pool having a bottom, using at least one of a stirring device, a brushing device and a wiping device, and an automatic moving arrangement configured for moving any of these devices in the pool. The stirring device comprises at least one wing having a longitudinal axis and at least two bottom contacting elements spaced from each other along the longitudinal axis of the wing so that, when the wing is in such close proximity to the bottom of the pool that the contacting elements contact its bottom, the contact of the wing with the bottom is prevented by the contacting elements.

### CROSS-REFERENCE TO RELATED APPLICATIONS

This application claims priority to Israel Patent Application No. 210376 filed on 30 Dec. 2010, the contents of which are incorporated herein, in their entirety by this reference.

### TECHNICAL FIELD

The presently disclosed subject matter relates to the field of pools containing various materials treated therein.

### BACKGROUND

The presently disclosed subject matter relates to pools containing various materials treated therein, such as pools used, e.g., for production of microorganisms, waste treatment, mixing and/or stirring various materials and chemicals, etc.

For example, in the field of production of microorganisms, it is well known to use specialized pools for carrying out processes related to the growth of. The pools are exposed to sunlight or to an artificial light source that enables photosynthesis to take place in the microorganisms. Water and carbon-dioxide mixed therein serve as a typical liquid in which the microorganisms are grown. Such microorganisms can be, for example, single-cell algae, such as: Nannochloropsis sp. Nannochloris sp. Phaeodactylum sp. Chlorococcum sp. Tetraselmis sp. Navicula lanzii; Navicula vim; Isochrysis sp. Chlorella sp. Spirulina sp. Aphnizomenon flos-aqua Amphora sp. Porphyridium sp. Hematococus sp. Donolealh sp. Diatom sp.

Various other types of microorganisms can also be grown this way. In the present description, the term ‘growth of microorganisms’ implies both their growth, i.e. enlargement, of each individual microorganism and their multiplication. The liquid itself can be any liquid wherein microorganisms can reside such as, for example, water, sea water, sewage, etc.

As microorganisms typically tend to sink and settle on the bottom of a pool in which they are treated, and as their amount of changes, it becomes necessary to stir the liquid in order to enable the sunk microorganisms to rise close to the surface of the pool and become exposed to light shining at the pool from above so that photosynthesis may take place. In addition, stirring the liquid allows to better mix its contents such as, for example, water and carbon-dioxide.

A typical well known method for stirring the liquid in the pool involves the use of one or more paddle wheels. Each paddle wheel is installed at one predetermined location in the pool and, as it rotates, produces a wave of moving liquid, that that travels along the pool. As it travels across the pool away from the paddle wheel the wave subsides, its energy is reduced, and areas of the pool away from the pedal wheel are less affected by it.

Some more advanced systems have also been developed for stirring liquid in a pool, for example, as disclosed in DE 2122891 and EP 261533.

### SUMMARY

The presently disclosed subject matter according to one aspect thereof relates to a system for treating materials, for example facilitating production of microorganisms, in a liquid in a pool having a bottom, comprising:
a stirring device comprising:
at least one wing having a longitudinal axis; and
at least contacting elements spaced from each other along the longitudinal axis of the wing; and
an automatic moving arrangement configured for moving the stirring device in the pool in a direction transverse to the longitudinal axis so that, when the wing is in such close proximity to the bottom of the pool that the contacting elements contact its bottom, the contact of the wing with the bottom is prevented by the contacting elements.

The presently disclosed subject matter according to its other aspect relates to a system for treating materials, for example facilitating production of microorganisms, in a liquid in a pool having a bottom, comprising:
a brushing device comprising:
at least one floating arrangement configured for floating on the liquid; and
at least one brush configured for brushing a bottom of the pool and being at least indirectly attached to the floating arrangement; and
an automatic moving arrangement configured for moving the brushing device in the pool.

A further aspect of the presently disclosed subject matter relates to a system for treating materials, for example facilitating production of microorganisms, in a liquid in a pool having a bottom, comprising:
a wiping device comprising:
at least one wiper; and
at least one weight increasing element configured to increase the clinging of the wiper to the bottom of the pool; and
an automatic moving arrangement configured for moving the wiping device in the pool.

The wiper can be made at least partially of a flexible material such as, for example, rubber and the like, allowing it to effectively push liquid across the bottom of the pool without damaging it (or any layer or liner covering the bottom).

In addition to the original wing, brush and wiper in the respective stirring, brushing or wiping device in any one or more of the above aspects of the presently disclosed subject matter, each such device can further comprise at least one of a wing, brush and wiper, which is mountable in such device in addition to or instead of its original element. In some examples, the further wing, brush or wiper can be mounted, onto the original element.

A still further aspect of the presently disclosed subject matter relates to a kit for treating materials, for example facilitating production of microorganisms, in a liquid in a pool having a bottom, comprising a floating arrangement and interchangeably attachable thereto at least two of the following three elements: a wing, a brush, and a wiper. In some examples, the kit can be part of a system further comprising an automatic moving arrangement configured for moving each of the interchangeably attachable elements in the pool.

At least one of the brushing device, the wiper and the kit in any one or more of the aspects of the presently disclosed subject matter, can further comprise at least two contacting elements spaced from each other along a longitudinal axis of the brush and/or the wiper. The contacting elements may be attached to edges of the wing, brush or wiper and/or at middle parts thereof.

The contacting elements in any one or more of the aspects of the presently disclosed subject matter can be wheels configured to rotate when in contact with the bottom of the pool. The wheels can be rotatable attached to the wing, brush or wiper. It should be understood that, while the wheels are spaced from each other along the longitudinal axis, they need not necessarily be positioned on the longitudinal axis or be rotatable with respect to it or parallel thereto.

The contacting elements in any one or more of the aspects of the presently disclosed subject matter can be sliding elements configured to slide on the bottom of the pool. The sliding elements can be, for example, in the form of non-rotating wheels, spheres, skis (in particular, with curved forward edges), etc.

The bottom of the pool in any one or more of the aspects of the presently disclosed subject matter can be in the form of a liner made of a liner material, and the contacting elements, when present, can have such a design as to cause no damage to the liner when rotating and/or sliding in contact therewith. Liner materials can be, for example, polyethylene (PE), polypropylene (PP), PVC, thermoplastic polyolefin (TPO), etc.

The stirring or wiping device in any one or more of the aspects of the presently disclosed subject matter can further comprise a floating arrangement configured for floating on the liquid, the wing or wiper being at least indirectly attached to the floating arrangement.

The floating arrangement can be attached to the wing, brush and/or wiper via an attachment mechanism. The attachment mechanism can fix the orientation and/or position of the wing and/or brush and/or wiper with respect to the floating arrangement. In one example, the attachment mechanism can comprise at least one rigid arm having ends, and connectors, wherein the connectors connect the ends of the rigid arm, respectively, to the floating arrangement and the wing and/or brush and/or wiper. In another example, the attachment mechanism can be flexible and may comprise cables and/or springs. The attachment mechanism, in some examples, can be adjustable, so as to be able to adjust the position and/or orientation of the wing, brush or wiper with respect to the floating arrangement.

The stirring, brushing or wiping device, or the kit, in any one or more of the aspects of the presently disclosed subject matter, can further comprise a rudder configured, when the device or kit is moving in the liquid, to stabilize movement of the device or kit through interaction with the liquid. In some examples, the rudder can be attached to the wing, brush or wiper and may be used to stabilize movement thereof. In further examples, the rudder is inclined at an angle other than 90 degrees, to a plane which is perpendicular to the longitudinal axis of the wing, brush or wiper and configured, when the device or kit is moving in the liquid, to push the device or kit or only the wing, brush or wiper, towards a side of the pool which is closest thereto.

The floating arrangement in any one or more of the aspects of the presently disclosed subject matter can have a leading portion defining a plane parallel to the longitudinal axis and extending upwardly and frontward of the remainder of the floating arrangement, to allow producing waves in the liquid when the floating arrangement is moved in the frontward direction.

The wing, brush and wiper in any one or more of the aspects of the presently disclosed subject matter, can have a longitudinal axis and the moving arrangement can be configured to move the corresponding element in a direction transverse to the longitudinal axis. In some examples, the movement direction can be inclined to the longitudinal axis of the moved element by at least 40 degrees, particularly, by at least 75 degrees, more particularly, by at least 85 degrees or perpendicular to the longitudinal axis.

When the pool has sides inclined relative to its bottom, in any one or more of the aspects of the presently disclosed subject matter, any of the stirring, brushing or wiping device, or the kit, can comprise a main portion extending along the longitudinal axis thereof and configured for corresponding interaction (i.e. stirring, sweeping, wiping) with liquid and microorganisms near the pool's bottom, and at least one secondary portion which is inclined with respect to the longitudinal axis and configured for interaction with liquid and microorganisms near the pool's sides.

The wiping device can have a weight increasing element(s) associated with it. The weight increasing element can be part of, attached to, or apply pressure to, the wiper. For example, the weight increasing element can be in the form of a layer(s) of material interposed between layers of the wiper or attached to the wiper. In another example, the weight increasing element(s) can be in the form of weights distributed in the wiper configured for pressing the wiper down, towards the bottom of the pool. The weight increasing element(s) can also be in the form of a crossbar(s) configured for providing rigidity to the construction of the wiper that helps it support its form

It should be understood that the wing, brush and wiper of any one or more of the above aspects can have various profiles, including non-standard profiles (i.e. shape in their cross-section taken perpendicular to their longitudinal axis). For example, any of the wing, brush or wiper can have a polygon profile, a partially or fully rounded profile, etc., or a combination of these forms. Furthermore, in some examples, the wing, brush or wiper can be segmented and at least some of these segments can have a longitudinal axis that is not aligned with the longitudinal axis of the whole wing, brush or wiper, accordingly.

In should be understood that the presently disclosed subject matter can used to obtain increased photosynthesis of microorganisms, and algae in particular, and/or for growing microorganisms, in particular to increase their biomass.

In the above aspects and examples of the presently disclosed subject matter, the floating arrangement can comprise one or more floating elements having buoyancy allowing them to stay on the surface of the liquid in the pool while supporting, at least partially, the weight of the wing and/or brush and/or wiper attached thereto. The floating arrangement can comprise of several different parts. In some examples, these parts can be, not only elements configured for floating, but also frame elements, connectors, bolts and screws, etc. Separated parts of the floating arrangement may be connected with rigid or non-rigid connections (e.g. cables and the like). Alternatively separated parts that comprise the floating arrangement may be completely separate from each other and connected to the wing, brush or wiper. The floating arrangement is configured to float on the liquid in the pool at least in a part thereof, and its structure is configured therefore. Exemplary shapes of the floating arrangement may include interconnected or separate pipe-like sections, boat-like shapes, elongated buoys, etc.

In the above aspects of the presently disclosed subject matter an automatic moving arrangement can, for example, comprise at least one of the following:
at least two pulleys and/or gears;
at least one motor, the motor configured for rotating at least one of the at least two pulleys and/or gears; and
at least one element selected from the group including a belt, a cable, a chain, a strap, linked elements etc., configured to be movable on the pulleys and/or gears.

At least a part of the moving arrangement, such as for example its cable/s, pulleys/gears, motor/s, etc., is configured to be located substantially above the liquid. The moving arrangement can generally be automatic, enabling an operator to turn it on or off, wherein in the ‘on’-state, the stirring, brushing or wiping device, or the wiper and/or brush and/or wing of the kit, is moved in the pool by the moving arrangement. In some examples, the operator can control the speed of movement. In other examples, the movement can be monitored by an automatic controller, such as a computer or a specialized control unit, having control of operation modes such as different movement speeds (for example, for day and night time and/or various steps of the production process planned to last for predetermined time periods) at different predetermined times that are controlled and/or monitored. The depth at which the wing, brush or wiper is disposed in the pool, can also be controlled by an operator or an automatic controller. When more than one of the wing, brush or wiper, are to be used at different stages of operation of the pool, the controller can also control their position and/or orientation with respect to the floating arrangement.

It should be noted that the moving arrangement can be configured to move any movable component(s) of the device or kit (wing, brush and/or wiper) across any portion of the pool and the movement can be unidirectional (e.g. only in the forward direction), bidirectional (e.g. back-and-forth), or multi-directional. For example, two pulleys can be used to move the movable components of the device or kit back-and-forth and/or continuously along a longitudinal direction of the pool. In another example, three or more pulleys can move any movable component(s) of the device or kit in a continuous loop across at least a portion of the pool with or without overlap of the areas of the pool covered by the component throughout the loop. In a large pool, several devices can be moved in a coordinated manner side by side across the pool to generate a greater wave in the liquid.

The stirring, brushing, or wiping device, or the kit, in accordance with the above aspects of the presently disclosed subject matter, is configured for being attachable to the at least one belt, cable or chain through a linking arrangement comprising at least one linking element. The linking arrangement can comprise, for example, one or more cables and/or rigid arms, straps, etc. and various connectors can be used to connect its ends to the device or kit and to the belt, cable, etc.

In some examples of the presently disclosed subject matter, the device or kit can further comprise sensor(s) for monitoring conditions or state of the liquid and/or microorganisms therein.

A still further aspect of the presently disclosed subject matter relates to a method of facilitating production of microorganisms in a liquid within a pool having a bottom, the method comprising at least one of the following three steps:
(a) stirring the liquid in the pool by at least one of the above-described stirring device, the brushing device and the wiping device;
(b) sweeping the microorganisms from the bottom of the pool by at least one of the above-described brushing device and the wiping device; and
(c) wiping the liquid on the bottom of the pool by the above-described wiping device.

The above steps can also be performed by the mentioned components that are parts of the kit as described above.

The method can comprise at least two of the steps (a), (b) and (c) above.

The method can further constitute a part of a process for producing microorganisms, the process comprising:
i. introducing and dispersing microorganisms in the liquid within the pool;
ii. growing the microorganisms in the liquid within the pool;
iii. extracting the microorganisms from the pool;
iv. cleaning the pool,
wherein
when the method comprises the step (a), this step is performed simultaneously with at least one of steps: (i), (ii) and (iii);
when the method comprises the step (b), this step is performed simultaneously with at least one of steps: (i), (ii) and (iii); and
when the method comprises the step (c), this step is performed simultaneously with step (iv).

In the above method, the microorganisms can be algae, in particular, single-cell algae selected from at least one of the following types: Nannochloropsis sp. Nannochloris sp. Phaeodactylum sp. Chlorococcum sp. Tetraselmis sp. Navicula lanzii; Navicula vim; Isochrysis sp. Chlorella sp. Spirulina sp. Aphnizomenon flos-aqua Amphora sp. Porphyridium sp. Hematococus sp. Donolealh sp. Diatom sp.

In accordance with a still further aspect of the presently disclosed subject matter, there is provided a pool including one or more devices as described above and/or operated by a method as described above.

The pool can have a bottom covered with a water-proof material, and the contacting elements referred to above can be configured to smoothly slide along the material without catching hold thereof.

### BRIEF DESCRIPTION OF THE DRAWINGS

In order to understand the disclosed subject matter and to see how it may be carried out in practice, embodiments will now be described, by way of non-limiting example only, with reference to the accompanying drawings, in which:

FIG. 1A schematically illustrates a stirring device according to one example of the presently disclosed subject matter, disposed in a pool

FIG. 1B is an enlarged view of the stirring device shown in FIG. 1A;

FIG. 2 is a schematic cross-sectional view of a stirring device according to another example of the presently disclosed subject matter, taken perpendicular to the longitudinal axis at the middle thereof;

FIG. 3 is a schematic front view of a stirring device according to a still further example of the presently disclosed subject matter;

FIGS. 4A to 4C schematically illustrate still further examples of a stirring device according to the presently disclosed subject matter;

FIG. 5 illustrates an example of a floating arrangement configured for use with a stirring device according to the presently disclosed subject matter;

FIG. 6 illustrates an example of a brushing device comprising a brush and a floating arrangement;

FIG. 7 illustrates an example of a wiping device;

FIG. 8 is a pool with a system according to the presently disclosed subject matter; and

FIGS. 9A to 9C are views of a pool with a system of according to the presently disclosed subject matter.

### DETAILED DESCRIPTION

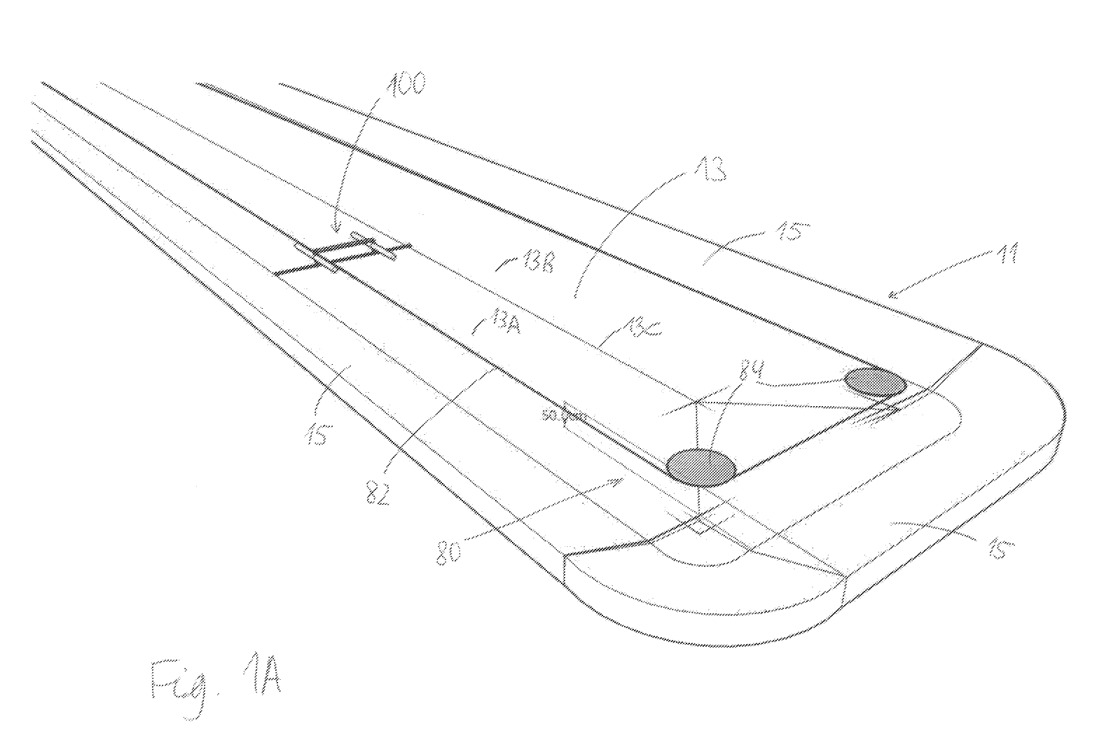

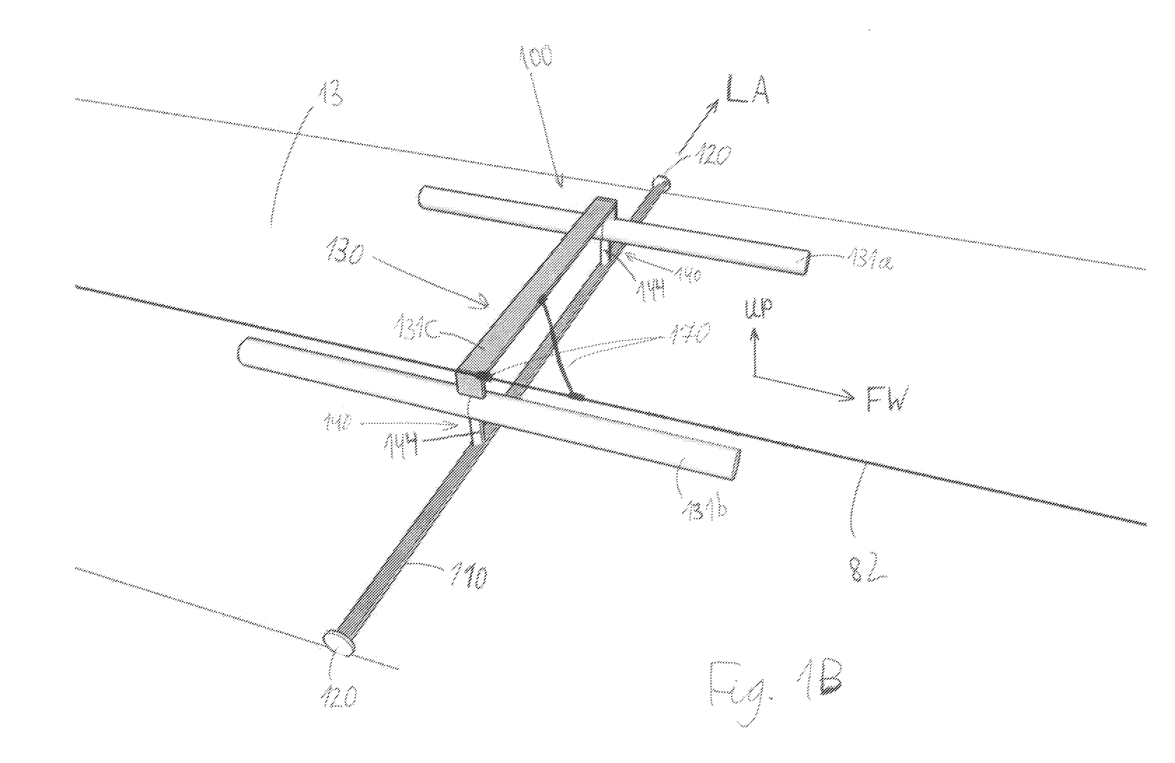

FIG. 1A is a schematic illustration of one example of a system 1 for treating materials in a liquid (not shown) in a pool 11 according to the presently disclosed subject matter. The system comprises a stirring device 100 and a moving arrangement 80 (shown partially), and the pool 11 has a bottom 13 and sloped sides 15.

The stirring device 100 better seen in FIG. 1B, comprises a wing 110 having a longitudinal axis LA, pool bottom contacting elements, a floating arrangement 130 which is connected to the moving arrangement 80, and an attachment mechanism 140 which connects the wing 110 to the floating arrangement 130.

The wing 110 is, generally, configured for stirring the liquid in the pool 11, as it is moved therein. In particular, the wing 110 is configured to increase pressure of the liquid below, and possibly in front, of it and lower the pressure of the liquid above, and possibly behind, it. The described effects on the pressure of the liquid induce an at least partially upwards directed flow of the liquid, i.e. in a direction UP from the bottom of the pool 11 to the surface of the liquid. The resulting liquid flow also causes flow of materials, such as for example microorganisms, in the corresponding upwards direction. The effects on the pressure of the liquid are typically localized to a volume of the liquid in vicinity of the wing. In some examples of the presently disclosed subject matter, however, the wing(s) 110 and/or its velocity can be configured to produce a significant wave(s) that travels in the liquid.

The wing 110 has profile(s), i.e. cross-sections of the wing 110 perpendicular to the longitudinal axis LA, the borders of which correspond to the outer surface(s) of the wing 110. This outer surface may be continuous, or comprise of any number of interconnected surfaces each of which can be planar, rounded, etc. The profile of the wing 110 can change along the longitudinal axis and individual sections, i.e. interconnected parts of the wing 110, or segments, i.e. non-connected parts of the wing 110, can have section or segment longitudinal axes that do not match the longitudinal axis of the whole wing 110.

While the wing's outer surface can be continuous or not, a profile of the wing 110 can be generally characterized as having a (leading) liquid moving surface 110l configured to face the liquid in the path of the wing 110 as it is moved in a frontward FW direction, a trailing surface 110t generally opposite the surface 110l, a rear portion 110re and a lower portion 110lw.

The pool bottom contacting elements of the stirring device 100 are elements configured, when in contact with the bottom 13 of the pool 11, to prevent contact between the wing 110 and the bottom 13. The contacting elements are also configured to contact the bottom 13 of the pool 11 without causing damage thereto. As such, when the stirring device 100 is moved in the pool 11 and the contacting elements contact the bottom 13, the contacting elements slide and/or roll on the bottom 13. Thus, the contacting elements can be sliding elements, such as skis, configured to slide and/or rotating elements, such as wheels and the like, configured to roll on the bottom 13 of the pool 11. The bottom contacting elements can be spaced from each other along the longitudinal axis, and in some examples may be positioned on the longitudinal axis or be rotatable with respect to it or parallel thereto. The contacting elements can be made, for example, of plastic and the like which means that they are relatively low weight, rounded and have little friction with the bottom 13 or a liner covering the bottom 13 of the pool 11, as well as being less prone to corrosion in the liquid.

In this example, the contacting elements are wheels 120 attached to the wing and rotatable about an axis (not shown) parallel to the longitudinal axis LA.

The floating arrangement 130 is configured to at least partially support the weight of the wing 110 and the attachment mechanism 140 in the liquid. The floating arrangement 130 can also be configured to generate a wave in the liquid in order to cause stirring thereof. It normally comprises at least one floating element whose length in the direction perpendicular to the longitudinal axis LA can be greater than its dimensions in other directions. In some examples, the floating arrangement can be combined with the attachment mechanism and even, in some further examples, the wing 110 and/or a brush 210 and/or a wiper 310 into a single unitary element.

It is noted that, in some examples of the device 100, the wing 110 can be attached directly to the moving arrangement 80, and a floating arrangement 130 may or may not be part of the stirring device 100.

In this example the floating arrangement 130 comprises two such floating elements in the form of tubular floats 131a and 131b and a middle element 131c connecting there-between. The middle element 131c is connected to upper portions of the tubular floats 131a and is disposed generally above them, so that it does not become fully or partially submerged in the liquid and increase the drag of the floating arrangement as it moves in the liquid. The floating arrangement 130 is connected to the linking arrangement 170 for its connection to the moving arrangement 80.

The attachment mechanism 140, that connects the wing 110 to the floating arrangement 130, can be configured to enable adjustment and fixating the distance, i.e. height, and/or the orientation of the wing 110 with respect to the floating arrangement 130. In FIG. 1B it can be seen that the attachment mechanism 140 comprises two similar rigid arms 144.

The moving arrangement 80 is configured for moving the stirring device 100 in the pool 11 along a predetermined path. As such, it can include, for example, a belt/cable/chain/etc. movable on pulleys/cog wheels/etc. rotated by a motor. In this example, the moving arrangement 80 includes a cable 82 guided by pulleys 84 and a motor (not shown).

The stirring device 100 is configured for being moved by the moving arrangement 80 in a frontward direction marked by FW in FIG. 1B, which is perpendicular to the longitudinal axis LA of the wing and to the upward direction UP of the pool, so as to stir the liquid in the pool 11, causing materials, such as microorganisms, located near or on the bottom of the pool 11 to rise closer to the surface of the liquid (for mixing with other materials, photosynthesis, etc.). Typically, the longitudinal axis LA is parallel to the bottom of the pool, when the latter is planar. It is not necessary that the longitudinal direction LA of the wing 110 be perpendicular to the frontward direction FW in which the stirring device 100 is moving, e.g. it can also be at any non-straight angle to the direction FW, provided that the wing is appropriately attached to the floating arrangement 130 when the latter is a part of the stirring device 100.

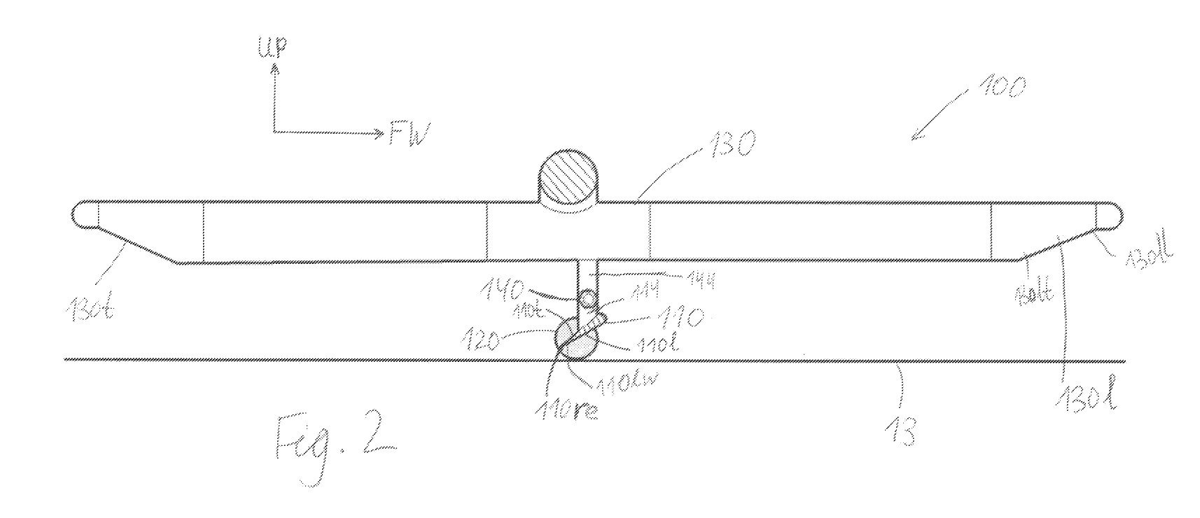

FIG. 2 shows a cross-sectional view of another exemplary stirring device 100 with its wing 110, taken perpendicular to the longitudinal axis LA of the wing at the middle thereof. The stirring device 100 of FIG. 2 differs from that shown in FIG. 1B in the orientation of its wing 110 and the construction of its floating arrangement 130. It can be seen that the wing 110 in FIG. 2 is inclined so that its rear portion 110re matches the lower portion 110lw of the wing 110 which is closest (typically at a distance of a few centimeters) to the bottom 13 of the pool, however, its contact therewith is prevented by the wheels 120 which extend lower than the rear portion 110re.

The floating arrangement 130 in FIG. 2 has front and rear faces 1301 and 110t, of which at least the front face 110l has a leading end 130ll and a trailing end 1301t, the front face 110l being defined by a plane parallel to the longitudinal axis LA and extending from the trailing end 130lt to the leading end 130ll generally in the upward UP and frontward FW directions. The front face 130l thus, when the stirring device 100 is moved in liquid, pushes on the liquid in front of the floating arrangement 130 and creates a wave that contributes to stirring of the liquid in the pool 11. The attachment mechanism 140 in FIG. 2 comprises the rigid arms 144 with holes for securing them with bolts to protrusions 114, also having holes, of the wing 110.

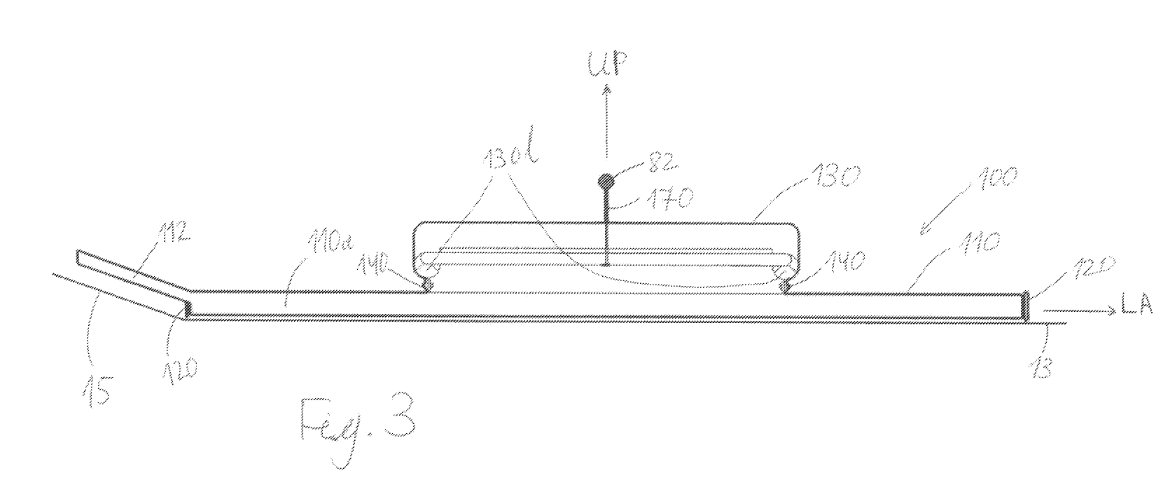

FIG. 3 shows a front view of another exemplary stirring device 100, which differs from that shown in FIG. 2 in that its wing 110 includes a main section 110a extending along the longitudinal axis LA parallel to the bottom 13 of the pool 11, and a secondary section 112 extending parallel to the inclined side 15 of the pool 13. Optionally, the wing 110 can have two secondary sections 112 attached at different sides of the main section 110a, and it can be used when the length of the wing 110 equals the entire width of the pool's 11 bottom, or when the pool 11 has an island and the length of the wing equals a distance between the sides of the pool and the island. Also shown is a linking arrangement 170 in the form of a cable 170c that connects a front section of the floating arrangement 130, in the middle thereof, to the cable 82, a cross-section of which is shown.

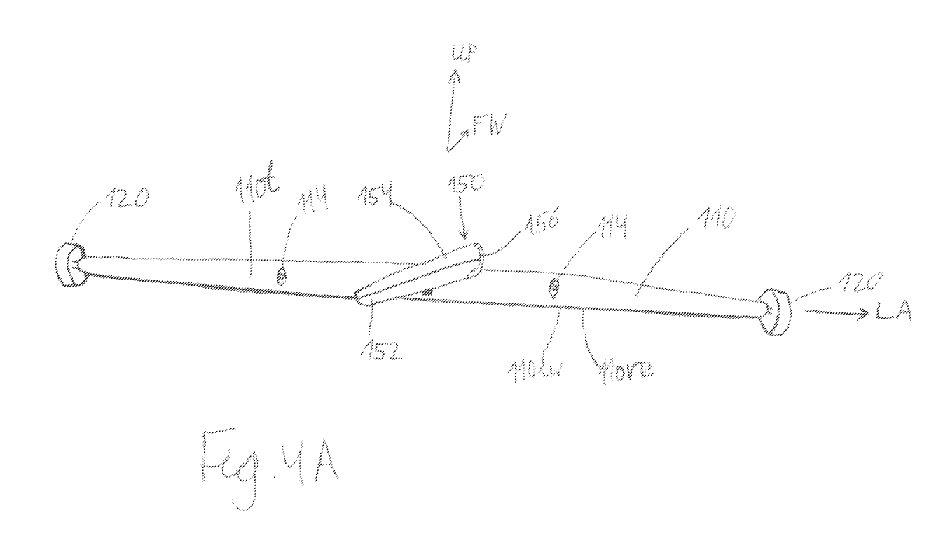

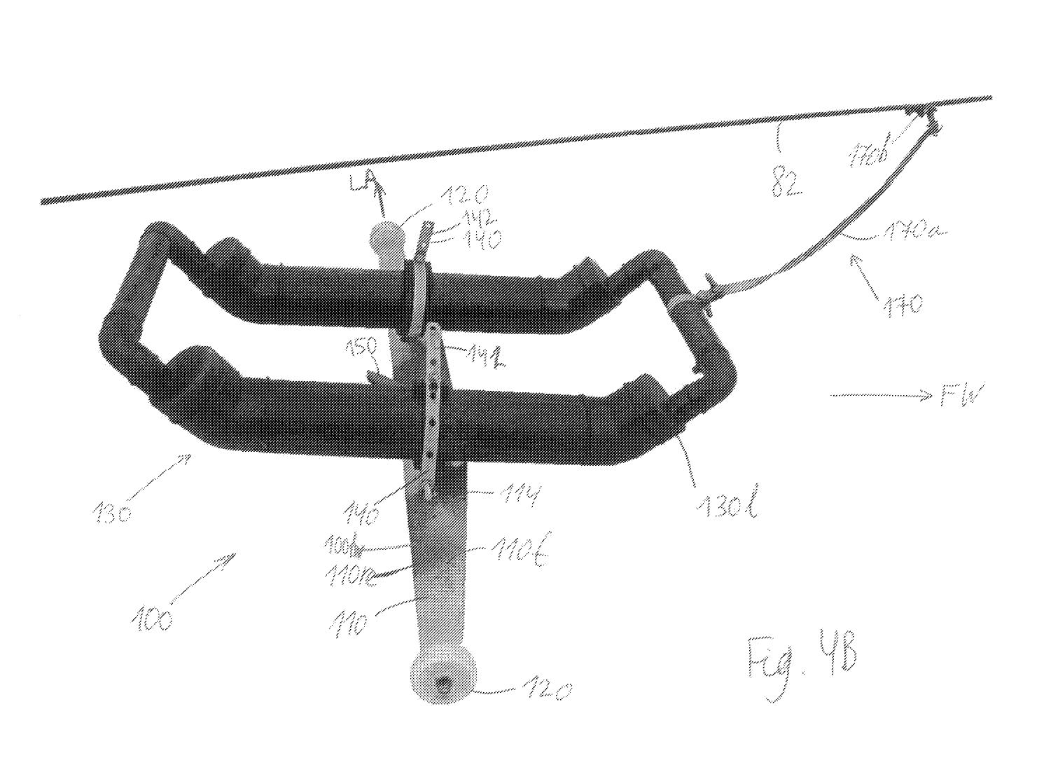

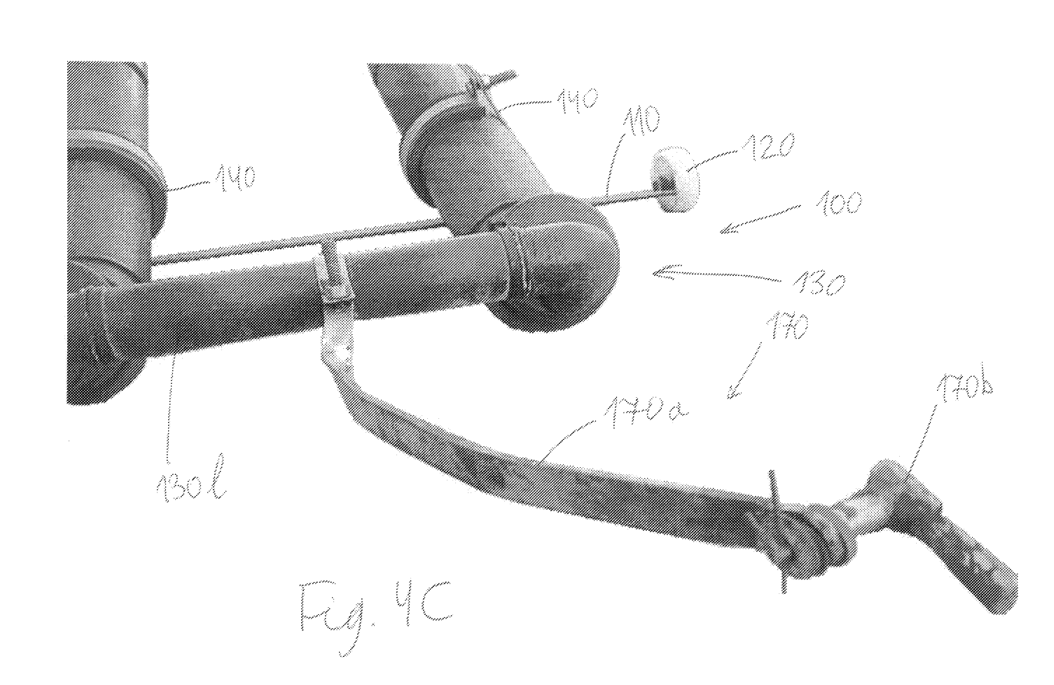

FIGS. 4A to 4C show a further example of the stirring device 100 and the wing 110 with the wheels 120 rotatably attached to it. In this example, the wing 110 comprises the protrusions 114 with holes, for connection to the attachment mechanism 140 or directly to the floating arrangement 130. In addition, a rudder 150 is mounted to the wing 110 and is fixable with respect thereto at various angles in a plane parallel to the liquid moving surface 110l of the wing 110 for stabilizing the movement of the device 100, when it is moving, through interaction with the liquid. This stabilization manifests itself in reduction of possible deviations of the device 100 from its predetermined path.

The rudder of this example comprises of a lower rudder portion 152 and an upper rudder portion 154 attachable to each other via a rudder connector 156. The rudder connector 156 enables customization of the rudder's 150 surface area by addition of upper rudder portions 154 of various sizes or by removal thereof. The rudder 150, causes the wing 110 to move along its predetermined path next to a side 15 of the pool 11 that is closest thereto. The rudder 150 also helps control the radius of turn of the wing 110 as it follows the cable 82 when it partially circles a pulley 84. Thus, the stirring device 100 can turn more smoothly and without applying undesirably high forces on the cable 82.

The floating arrangement 130 in this example comprises several interconnected pipe sections. The attachment mechanism 140 connects between the floating arrangement 130 and wing 110, and comprises metal strips 142 having holes along their longitudinal dimension which can be associated with the holes of protrusions 114 to enable both types of holes to be secured by bolts. The linking arrangement 170, shown in FIGS. 4B and 4C, comprises a rigid arm 170a and a cable connector 170b for connection to the cable 82.

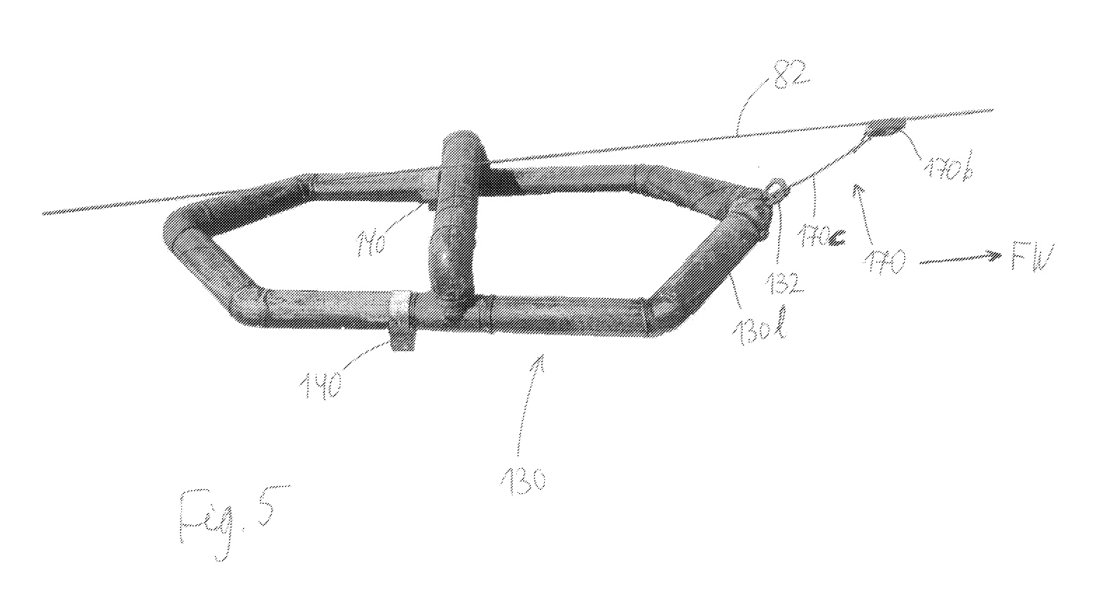

FIG. 5 shows another example of the floating element 130 floating on the liquid, which hides additional elements located below it. Also shown is the linking arrangement 170 comprising the cable connector 170b and the linking cable 170c. The cable 170c is attached to a connector 132 of the floating arrangement 130. A portion of the attachment mechanism 140 is also shown.

All features described above with respect to specific examples can be used in other examples in any combination.

Some examples of a brushing device 200 and a wiping device 300 will now be described, and afterwards exemplary structures of the moving arrangement 80 will be shown. It is noted that features described in specific examples of the devices 100, 200 and 300 can also be used in any combination, while the wing 110, brush 210 and wiper 310 may substitute each other without necessarily requiring changes in any of the other features or readily foreseeable functions of the devices 100, 200 and 300. It is also noted that, more than one wing 110, brush 210, or wiper 310 and combinations of these can be attached the floating arrangement 130 directly or by the same or different attachment mechanism(s) 140.

The brushing device 200 is, generally, configured to sweep material from the bottom of the pool 11 and can also be used to stir liquid in the pool 11. It comprises at least the brush 210 and the floating arrangement 130, and is connected to the moving arrangement 80.

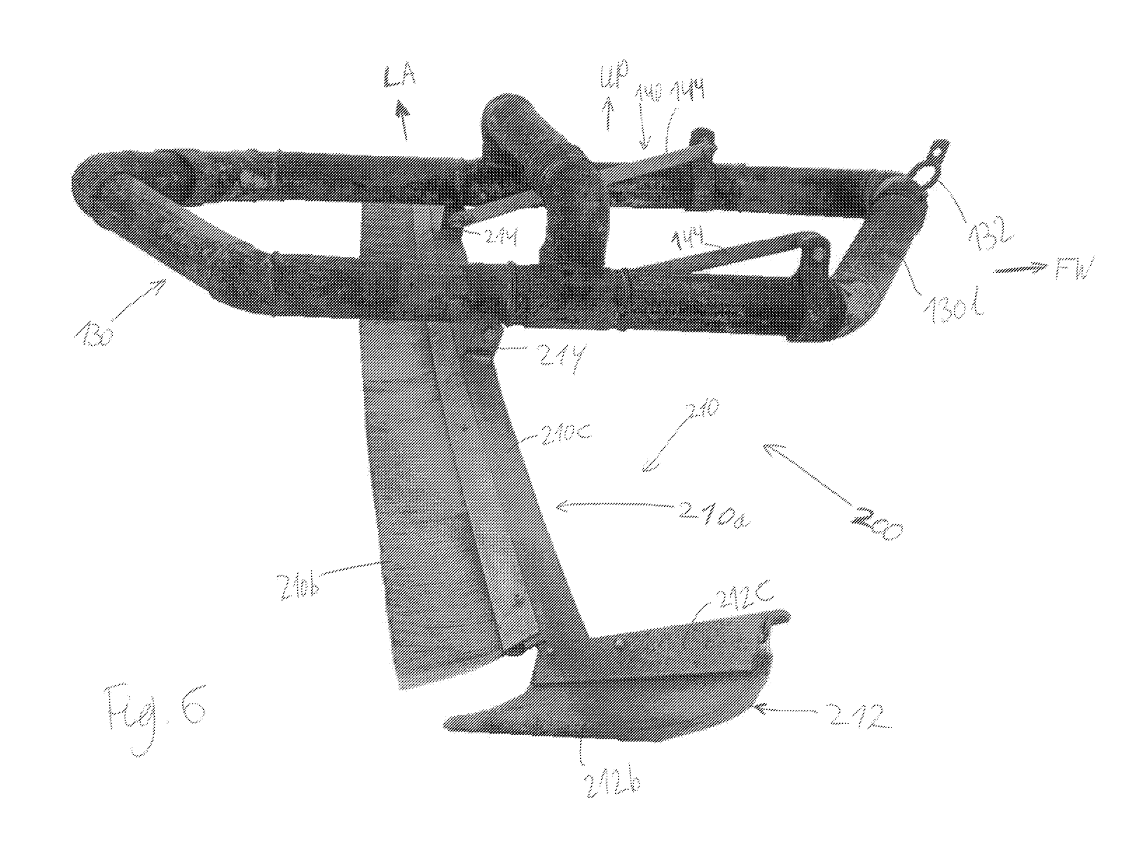

FIG. 6 shows an example of a brushing device 200. The device 200 comprises a brush 210 and a floating arrangement 130. The brush 210 comprises a main section 210a and a secondary section 212 for sweeping, respectively the bottom 13 and the sides 15 of the pool 11. The brush 210 further has main and secondary brushing portions 210b and 212b and main and secondary rigid brush frame portions 210c and 212c. The rigid arms 144 of the attachment mechanism 140 are, in this example, attachable by bolts to the floating arrangement 130, and to the brush 210 through protrusions 214 with holes on the main rigid brush frame portion 210c. Optionally, the brush 210 can be equipped with the wheels 120 positioned along the longitudinal axis of the brush and preventing contact between the main rigid brush frame portion 210c and the bottom 13 of the pool 11.

The wiping device 300 is, generally, configured to wipe liquid from the bottom of the pool 11, and can also be used to stir liquid in the pool 11 and/or directly (i.e. physically) sweep materials from the bottom 13. It comprises at least the wiper 310 and a weight increasing element, for increasing clinging of the wiper 310 to the bottom 13 of the pool 11, and is connected to the moving arrangement 80.

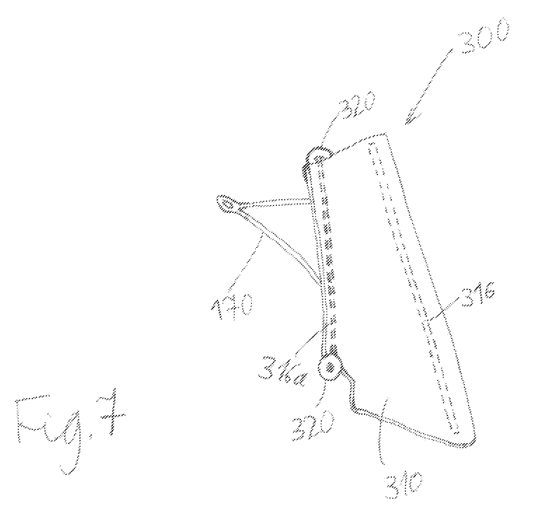

FIG. 7 shows an example of a wiping device 300. The device 300, in this example, does not include the floating arrangement 130. The linking arrangement 170 is used to connect a wiper 310 directly to the cable 82. The wiping device 300 comprises: the wiper 310 and a weight increasing element 316 in the form of a metal crossbar. The wiping device 300 further comprises: wheels 320 mounted rotatable to a crossbar 316a which further serves as an additional weight increasing element, and a linking arrangement 370 for attaching the device 300 to the cable 82.

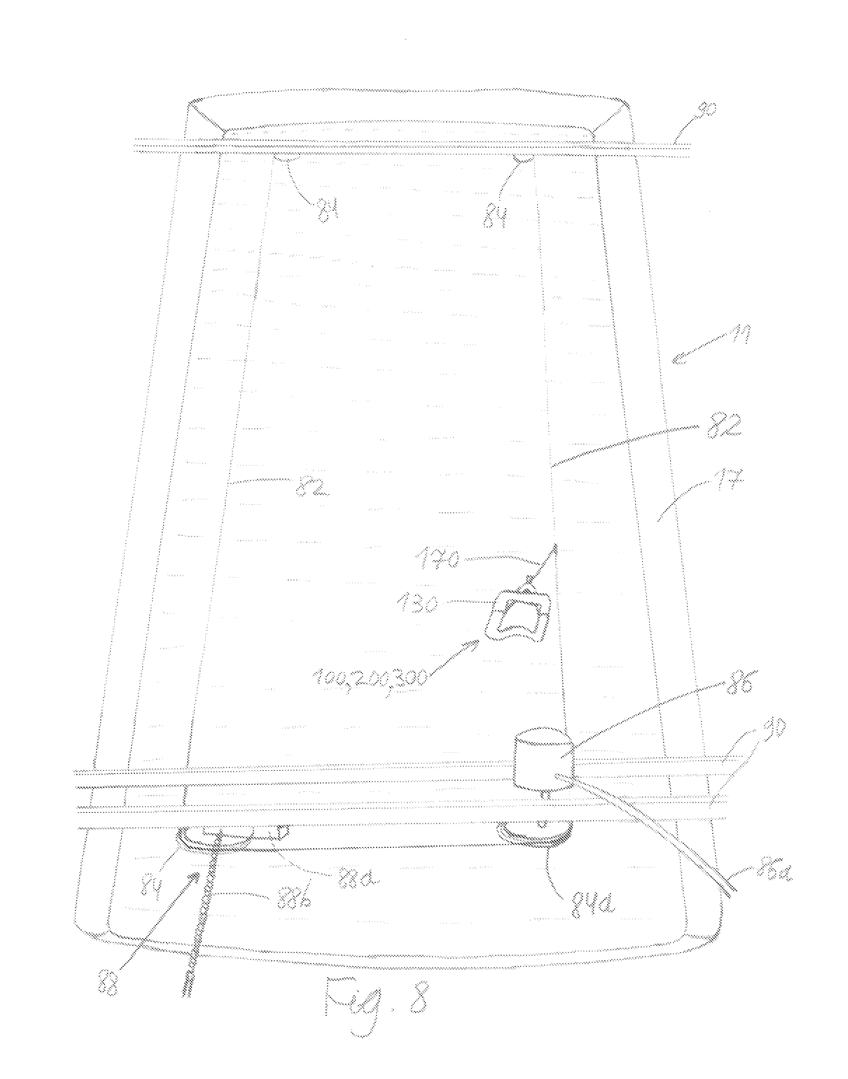

FIG. 8 shows the pool 11 with liquid therein. The pool's 11 bottom 13 and sides 15 are covered by a liner 17. The linking arrangement 170 connects the floating arrangement 130 to the cable 82 which is movable by a pulley 84a rotatable by the motor 86. The cable 82 is further guided by the pulleys 84 which rotate due to interaction, i.e. friction, with the cable 82 as it is moved by the pulley 84a. Fuel line 86a supplies fuel to the motor 86. In this example, the moving arrangement can be turned on or off by activating or deactivating a fuel pump (not shown) that pumps fuel to the motor 86. The pulleys 84 and 84a and the motor 86 are supported by cross-beams 90. A stretching mechanism 88 is also shown and comprises: an arm 88a supporting one of the pulleys 84 and freely rotatable with respect to the overhead beam 90, and a spring 88b attached between a stationary point outside the pool and a free end of the arm 88a. The stretching mechanism 88 is used to keep tension and provides adjustment for slightly different lengths, of the cable 82.

When any of the devices described above is used in the pool 11, being attached to the linking arrangement 170 and moved by the moving arrangement 80, the wheels 120 protect the bottom 13 of the pool 11 from damage that might be caused to it by the wing 110 if it would not have such wheels. In particular, when covered by the liner 17, which can be prone to have folds, it can be easily torn by a solid object dragged on it.

The use of liners in pools of the kind in which the devices described above are intended to be used, is known since such liners are very effective means to isolate the bottom and sides of large ground dug pools, which can cover large areas and be used to grow large amount of microorganisms, significantly more than other types of pools which typically require more elaborate construction. Typically for large ground dug pools, the vertical to horizontal measures ratio of the sides 15 of the pool—the measures corresponding respectively to the depth of the pool at the edges of its bottom and to the width of the sides of the pool, is in a range of 1:4 to 1:5. The bottom 13 of the pool 11 typically has two halves, marked 13A and 13B in FIG. 1A, extending along the length of the pool between the sides 15 and a center-line 13c where the two halves 13A and 13B meet. Each half 13A or 13B is typically inclined at an angle of about 2 degrees, declining from the sides of the pool 15 until the center-line 13c wherein the pool has its greatest depth.

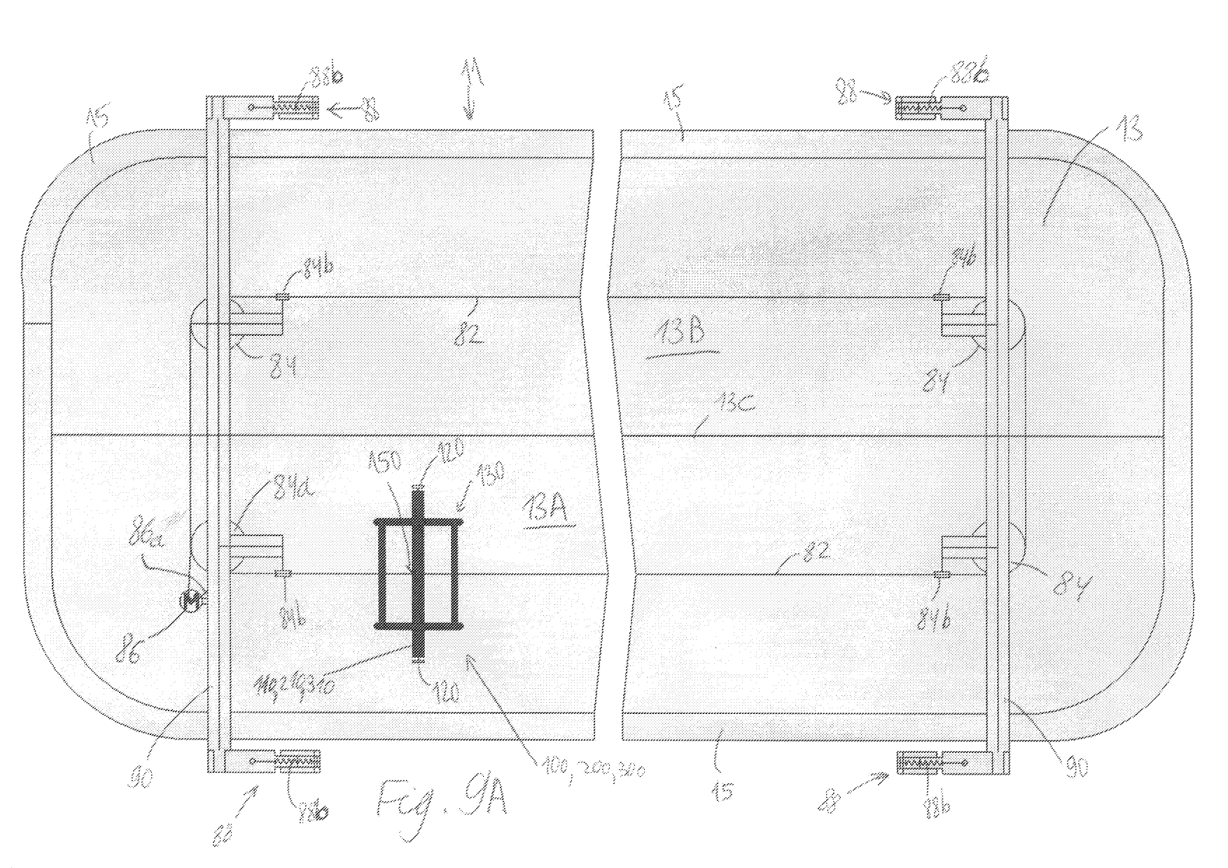

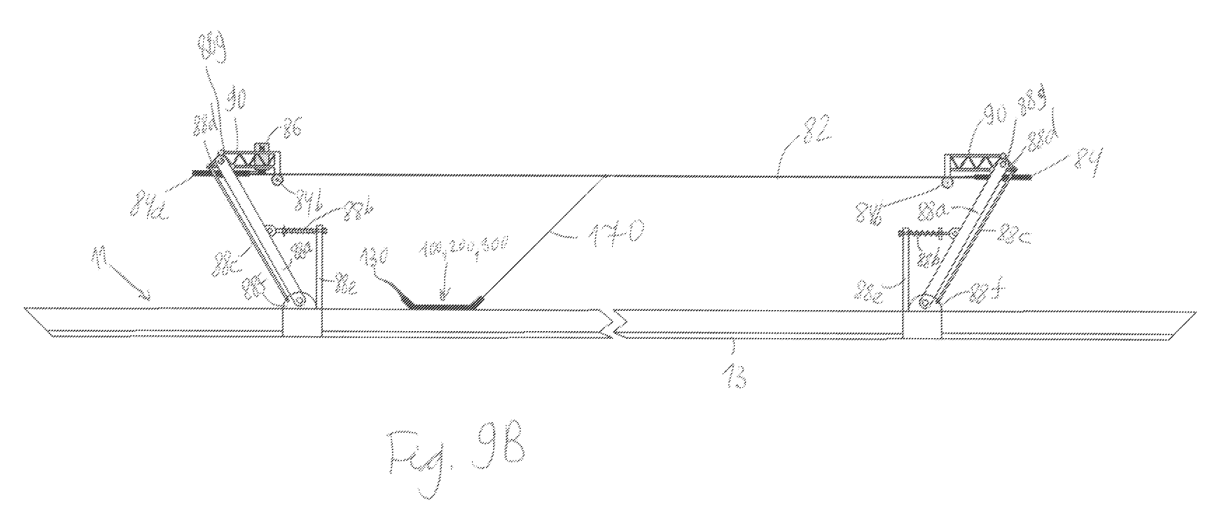

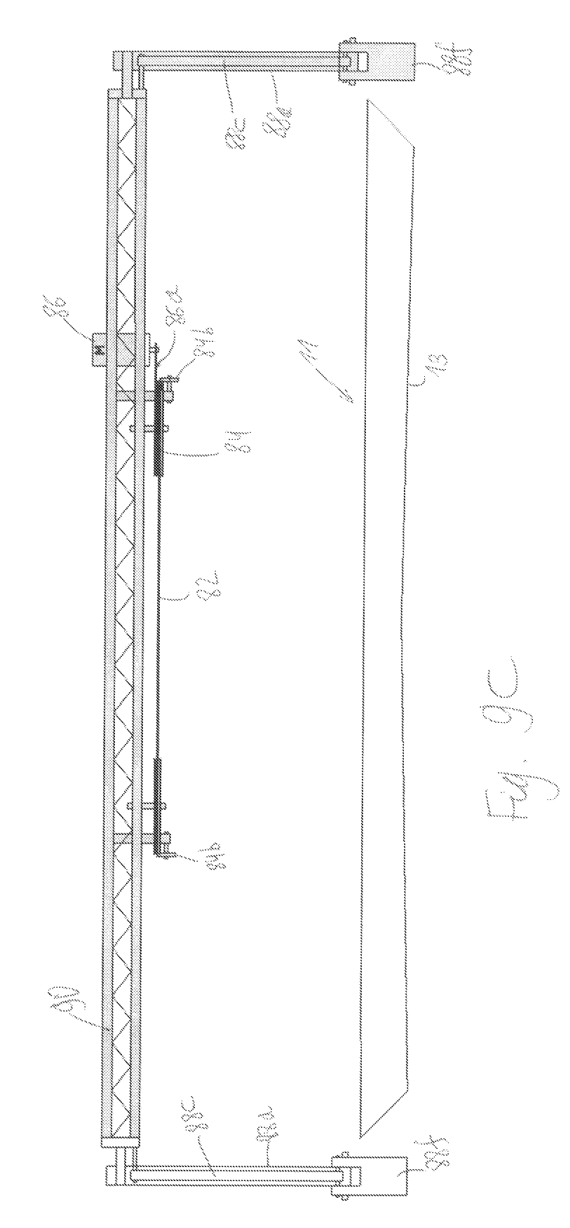

FIGS. 9A to 9C show a top, right and front views of the pool 11 with the system as described above, including devices 100, 200, 300 positioned in the pool 11. The device 100, 200, 300 is connected by the linking arrangement 170 to the cable 82. The cable 82 is moved by the pulley 84a which is rotated by belt 86a which is connected to the motor 86. The pulleys 84, 84a and small pulleys 84b, which are aligned in a vertical plane, are used to guide the cable 82 as it moves in a closed loop motion across the pool 11. The stretching mechanism 88 generally comprises: arms 88a, springs 88b, cables 88c, triangular frames 88d, vertical poles 88e, bases 88f and axles 88g. Each of the arms 88a is rotatable mounted to one of the bases 88f, vertical poles 88e are static and positioned near respective bases 88f. The spring 88b is disposed between the pole 88e and the arm 88a and can push or pull the arm 88a away from or towards the pole 88e. The arm 88a is connected at its upper edge to the axle 88g positioned inside the triangular frame 88d and supporting it. The triangular frame 88d is attached to the cross-beams 90 which support the pulleys 84, 84a, 84b and the motor 86. In operation, the stretching mechanism 88 provides tension to the cable 82 as it is indirectly pulled in opposing directions by the arms 88a. Orientation of the cross-beams 90, which in this case should be maintained horizontal regardless of the angle of arms 88a, is adjustable, manually or automatically, by controlling the length of the cables 88c that are connected to one corner of the triangular frames 88d causing them to pivot with respect to the axles 88g that support them and correspondingly changing the orientation of the cross-beams 90 attached thereto.

In a typical process of microorganism treatment and/or production, the microorganisms are first introduced into the pool. Afterwards, the microorganisms are grown and/or treated and later on the liquid in the pool is evacuated and the microorganisms may be extracted. Subsequently, the pool is cleaned.

The devices 100, 200, 300 can be used alone or in combination at some or all of the above stages. For example, as the wing 110 stirs the liquid in the pool, it can be used both disperse the microorganisms in the pool as they being introduced and to subsequently stir the liquid causing the microorganisms to rise from the bottom of the pool to become exposed to light and/or to better mix with the constituents of the liquid. The wing can also be used to move the liquid in the pool so as to hasten its eviction at the extraction and evacuation stage.

The brush 210 can be used at any of the stages to both stir the liquid in the pool, sweep microorganisms directly from the bottom of the pool causing them to rise upwards in the liquid and mix therewith, help extract the microorganisms from the pool by sweeping them towards extraction openings or tubes and subsequently to clean the pool.

The wiper 310 can also be used at any of the stages, and subsequently to wipe the fluid from the bottom and sides of the pool as part of the cleaning stage.

It should be noted that the wheels, brush and wiper need not be in physical contact with the bottom and/or sides of the pool throughout the entire process. For example, they may be floated by the floating arrangement 130 above the bottom and sides of the pool at some point(s) during the process.

The system and devices described above can be used to treat and/or grow microorganisms in shallow water, only a few centimeters deep, through the use of either of the wing 110, brush 210, wiper 310 and combinations thereof. For example, a small amount of microorganisms can be introduced into a large pool with a low level of liquid therein, and after they are dispersed and as they grow, the level of the liquid can be gradually raised.

The devices 100, 200, 300 and combinations thereof can also be effective in extracting the microorganisms from the liquid. Whereas, in a standard pool the liquid is typically evacuated with the microorganisms, in the present case the microorganisms may be allowed to settle on the bottom of the pool, after which a significant portion of the liquid above the microorganisms can be evacuated, and subsequently the devices 100, 200 and/or 300 can be used to remove the remaining liquid near the bottom of the pool—with the microorganisms in significantly higher concentration.

Further systems according to the presently disclosed subject matter and combinations thereof can also be effective in treating and/or growing different types of microorganisms with different properties including weight, density, concentration in the liquid, etc. For example, microorganisms having high density and/or are too heavy to be stirred by prior art systems.

1. A system for treating materials in a liquid in a pool having a bottom, comprising:
a stirring device including:
at least one wing having a longitudinal axis; and
at least two bottom contacting elements spaced from each other along the longitudinal axis of the wing; and
an automatic moving arrangement configured for moving the stirring device in the pool in a direction transverse to the longitudinal axis so that, when the at least one wing is in such close proximity to the bottom of the pool that the at least two bottom contacting elements contact its bottom, contact of the at least one wing with the bottom is prevented by the at least two bottom contacting elements.

2. The system according to claim 1, wherein the stirring device further comprises at least one of the following: an additional wing, a wiper, or a brush that is mountable therein in addition or instead of the at least one wing.

3. The system according to claim 1, wherein at least one of the contacting elements is a wheel configured to rotate when in contact with the bottom of the pool and/or a sliding element configured to slide on the bottom of the pool.

4. The system according to claim 1, wherein the stirring device further comprises a floating arrangement configured for floating on the liquid, the at least one wing being at least indirectly attached to the floating arrangement via an attachment mechanism, which fixes the orientation and/or position of the at least one wing with respect to the floating arrangement.

5. The system according to claim 1, wherein the stirring device further comprises a rudder configured, when the stirring device is moving in the liquid, to stabilize movement of the stirring device through interaction with the liquid.

6. The system according to claim 5, wherein at least a portion of the rudder is inclined at an angle other than 90 degrees, to a plane which is substantially perpendicular to the longitudinal axis of the at least one wing and configured, when the stirring device is moving in the liquid, to push the stirring device towards a side of the pool that is closest thereto.

7. The system according to claim 4, wherein the floating arrangement has a leading portion defining a plane substantially parallel to the longitudinal axis and extending upwardly and frontward of the remainder of the floating arrangement, to allow producing waves in the liquid when the floating arrangement is moved in the frontward direction.

8. A system for treating materials in a liquid in a pool having a bottom, comprising:
a brushing device comprising:
at least one floating arrangement configured for floating on the liquid; and
at least one brush configured for brushing a bottom of the pool and being at least indirectly attached to the floating arrangement; and
an automatic moving arrangement configured for moving the brushing device in the pool.

9. The system according to claim 8, wherein the brushing device further comprises at least two bottom contacting elements.

10. The system according to claim 8, wherein the brushing device further comprises a rudder configured, when the brushing device is moving in the liquid, to stabilize movement of the brushing device through interaction with the liquid.

11. The system according to claim 8, wherein the floating arrangement has a leading portion defining a plane substantially parallel to a longitudinal axis of the brush, extending upwardly and frontward of the remainder of the floating arrangement, to allow producing waves in the liquid when the floating arrangement is moved in the frontward direction.

12. A system for treating materials in a liquid in a pool having a bottom, comprising:
a wiping device comprising:
at least one wiper; and
at least one weight increasing element configured to increase the clinging of the wiper to the bottom of the pool; and
an automatic moving arrangement configured for moving the wiping device in the pool.

13. The system according to claim 12, wherein the wiper has a longitudinal axis and wherein the wiping device further comprises at least two wheels spaced from each other along the longitudinal axis and configured to rotate when in contact with the bottom of the pool.

14. The system according to claim 12, wherein the wiping device further comprises a floating arrangement configured for floating on the liquid, the wiper being at least indirectly attached to the floating arrangement.

15. A kit for treating materials in a liquid in a pool having a bottom, comprising a floating arrangement and interchangeably attachable thereto at least two of the following three elements: a wing, a brush, or a wiper.

16. The kit according to claim 15, wherein each interchangeably attachable element has a longitudinal axis, and wherein the kit further comprises at least two bottom contacting elements spaced from each other along the longitudinal axis.

17. The kit according to claim 15, where at least one of the interchangeably attachable elements is further associated with a rudder configured, when the element is moving in the liquid, to stabilize its movement through interaction with the liquid.

18. The kit according to claim 15, wherein each interchangeably attachable element has a longitudinal axis, and the floating arrangement has a leading portion defining a plane substantially parallel to the longitudinal axis and extending upwardly and frontward of the remainder of the floating arrangement, to allow producing waves in the liquid when the floating arrangement is moved in the frontward direction.

19. The kit according to claim 15, being part of a system further comprising an automatic moving arrangement configured for moving each of the interchangeably attachable elements in the pool.

20. A method of facilitating production of microorganisms in a liquid within a pool having a bottom, comprising at least stirring the liquid in the pool by at least the stirring device of the system of claim 1.

21. The method according to claim 20, wherein the microorganisms are single-cell algae selected from at least one of the following types:
Nannochloropsis sp., Nannochloris sp., Phaeodactylum sp., Chlorococcum sp., Tetraselmis sp., Navicula lanzii, Navicula vim, Isochrysis sp., Chlorella sp., Spirulina sp., Aphnizomenon flos-aqua, Amphora sp., Porphyridium sp., Hematococus sp., Donolealh sp., or Diatom sp.
